(19) United States
(12) Patent Application Publication
(10) Pub. No.: US 2004/0147478 A1
(43) Pub. Date: Jul. 29, 2004

(54) Combination chemotherapy

(75) Inventor: Ronald Lynn Merriman, Ann Arbor, MI (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/713,337

(22) Filed: Nov. 14, 2003

Related U.S. Application Data
(60) Provisional application No. 60/426,717, filed on Nov. 15, 2002.

(51) Int. Cl. A61K 31/70

(52) U.S. Cl. 514/46

(57) ABSTRACT

This invention relates to a method for treating cancer utilizing a combination of known oncolytic agents. Specifically, this invention relates to a method for treating cancer utilizing a combination of a MEK inhibitor and capecitabine.

This application is a U.S. utility application which claims the benefit of priority to U.S. provisional application Serial No. 60/426,717 filed Nov. 15, 2002.

### FIELD OF THE INVENTION

The present invention relates to a method for treating cancer utilizing a combination of known oncolytic agents. Specifically, this invention relates to the combination of a MEK inhibitor and capecitabine.

### BACKGROUND OF THE INVENTION

Cancer chemotherapy has advanced dramatically in recent years. Many tumors can be effectively treated utilizing compounds, which are either naturally occurring products or synthetic agents. Cancer chemotherapy can entail the use of a combination of agents, generally as a means to reduce the toxic effects of the individual agents when used alone, and in some instances because the combination has greater therapeutic effects than when either agent is used alone.

In tumors, the Ras-Raf-MEK-ERK pathway appears to be the single most important pathway for the transmission of mitogenic signals from the plasma membrane to the nucleus. Activated raf activates by phosphorylation the signaling kinases MEK1 and MEK2 (MEK 1/2). These are dual-specificity kinases that activate the ERK family kinases, ERK1 and ERK2, by phosphorylation of both threonine and thyrosine. ERK activation results in phosphorylation and activation of ribosomal S9 kinase and transcription factors, such as c-Fos, c-Jun and c-Myc, resulting in the switching on of a number of genes involved in proliferation. A variety of growth factors, such as the erbB family, PDGF, FGF and VEGF, transmit signals through the Ras-Raf-MEK-ERK pathway. In addition, mutations in ras proto-oncogenes can result in constitutive activation of this pathway. Ras genes are mutated in approximately 30% of all human cancers, and the frequencies of ras mutations are particularly high in colon and pancreatic cancers (50% and 90%, respectively). Because of their downstream position from various mitogenic factors, MEK 1 and 2 have a central role in the transmission of proliferative signals from the plasma membrane to the nucleus. This makes these proteins a potentially better target for cancer therapy because their inhibition would abrogate a number of different signaling pathways. Therefore, a MEK inhibitor may be active against a broad range of cancers, such as, but not limited to, breast, colon, lung, ovarian and pancreatic cancers.

2-(2-Chloro-4-iodo-phenylamino)-N-cyclopropylmethoxy-3,4-difluorobenzamide, also known as CI-1040 is a potent and highly selective inhibitor of both Mek isoforms, MEK1 and MEK 2. Inhibition of MEK activity by CI-1040 results in a significant decrease in the levels of phosphorylated ERK1 and ERK2. This decrease produces a G1 block and impairs the growth of tumor cells, both in culture and in mice. CI-1040 has demonstrated anticancer activity against a broad spectrum of tumor types, including those of colon and pancreatic origin (Sebolt-Leopold J., et al, Blockade of the MAP kinase pathway suppresses growth of colon tumors in vivo. Nature Med 1999; 5:810-16; and Sebolt-Leopold J S, Summary of the preclinical pharmacology of CI-1040. RR 700-00156. Jun. 27, 2000.).

CI-1040 is described in PCT Publication No. WO 99/01426, which is incorporated herein by reference for its teaching of how to make CI-1040, how to formulate it into dosage forms, and how to use it for chronic oral treatment of solid tumors, such as breast, colon, prostate, skin and pancreatic cancers. CI-1040 is also described in U.S. Pat. No. 6,251,943 for use in the treatment or prevention of septic shock.

N-[(R)-2,3-Dihydroxy-propoxy]-3,4-difluoro-2-(2-fluoro-4-iodo-phenylamino)-benzamide (“Compound A”) is a potent and highly selective, inhibitor of MEK1/2, which significantly inhibits the phosphorylation of ERK1 and ERK2. Compound A is described in PCT Publication No. WO 02/06213, which is incorporated herein by reference for its teaching of how to make it, how to formulate it into dosage forms, and how to use it for chronic oral treatment of solid tumors, such as breast, colon, prostate, skin and pancreatic cancers. It is more potent and metabolically more stable than its predecessor, CI-1040.

Capecitabine is a fluoropyrimidine carbamate with antineoplastic activity. It is an orally administered systemic prodrug of 5′-deoxy-5-fluorouridine (5′-DFUR) which is converted to 5-fluorouracil. The chemical name for capecitabine is 5′-deoxy-5-fluoro-N-[(pentyloxy)carbonyl]-cytidine. It is marketed in the United States as Xeloda™ (Roche Laboratories). It is indicated for the treatment of patients with metastatic breast cancer and colorectal tumors. It generally is administered for 14 days, followed by a 7-day rest period during each 21-day cycle. Capecitabine is described in U.S. Pat. No. 5,472,949.

### SUMMARY OF THE INVENTION

The present invention provides a method for treating cancer in a patient in need of such treatment, the method comprising administering to the patient a combination of a therapeutically effective amount of a MEK inhibitor and a therapeutically effective amount of capecitabine.

The combination of the present invention may be administered simultaneously, the MEK inhibitor may be administered before capecitabine or capecitabine may be administered before the MEK inhibitor.

According to the combination or method of the present invention, the MEK inhibitor may be CI-1040 or N-[(R)-2,3-dihydroxy-propoxy]-3,4-difluro-2-(2-fluoro-4-iodo-phenylamino)-benzamide.

Additionally, the method of the present invention also provides that CI-1040 or N-[(R)-2,3-dihydroxy-propoxy]-3,4-difluro-2-(2-fluoro-4-iodo-phenylamino)-benzamide may be administered before capecitabine or capecitabine may be administered before CI-1040 or N-[(R)-2,3-dihydroxy-propoxy]-3,4-difluro-2-(2-fluoro-4-iodo-phenylamino)-benzamide.

The present invention also provides a method for treating cancer in a patient in need of such treatment, the method comprising administering to the patient a therapeutically effective amount of capecitabine followed by administering to the patient a therapeutically effective amount of CI-1040.

Additionally provided by the present invention is a method for treating cancer in a patient in need of such treatment, the method comprising the steps of administering to the patient a therapeutically effective amount of N-[(R)-2,3-dihydroxy-propoxy]-3,4-difluro-2-(2-fluoro-4-iodo-phenylamino)-benzamide followed by administering to the patient a therapeutically effective amount of capecitabine.

An embodiment of the present invention provides a pharmaceutical composition comprising capecitabine, CI-1040 and a pharmaceutically acceptable carrier.

Another embodiment of the present invention provides a pharmaceutical composition comprising capecitabine, N-[(R)-2,3-dihydroxy-propoxy]-3,4-difluro-2-(2-fluoro-4-iodo-phenylamino)-benzamide and a pharmaceutically acceptable carrier.

Another aspect of the invention is a kit comprising in one compartment a dosage of CI-1040, and in another compartment a dosage of capecitabine. For example, the invention includes: (a) a blister pack containing separate formulations of each active, such as a tablet or capsule form of CI-1040 and a tablet form of capecitabine; and (c) a kit with separate formulations of each active packaged together in a box with instructions for combination administration.

### DETAILED DESCRIPTION OF THE INVENTION

The patient to be treated according to this invention includes any warm-blooded animal, such as, but not limited to human, horse, dog, guinea pig, or mouse. For example, the patient is human. Those skilled in the medical art are readily able to identify individual patients who are afflicted with cancer and who are in need of treatment. Typical cancers to be treated according to this invention include, but are not limited to, brain, breast, lung, such as non-small cell lung, ovarian, pancreatic, prostate, renal, colon, cervical, acute leukemia, gastric cancer, melanoma, and other cancers susceptible to treatment with capecitabine and/or MEK inhibitors, such as CI-1040 and Compound A. The term “treatment” for the purpose of the present invention includes treatment, inhibition, control, prophylaxis or prevention, amelioration or elimination of a named condition, such as cancer, once the named condition has been established.

CI-1040 and Compound A are selective MEK 1 and MEK 2 inhibitors. Selective MEK 1 or MEK 2 inhibitors are those compounds which inhibit the MEK 1 or MEK 2 enzymes without substantially inhibiting other enzymes such as MKK3, ERK, PKC, Cdk2A, phosphorylase kinase, EGF and PDGF receptor kinases, and C-src. In general, a selective MEK 1 or MEK 2 inhibitor has an IC₅₀ for MEK 1 or MEK 2 that is at least one-fiftieth (1/50) that of its IC₅₀ for one of the above-named other enzymes. A selective inhibitor may have an IC₅₀ that is at least 1/100, 1/500, or even 1/1000, 1/5000 or less than that of its IC₅₀ for one or more of the above-named enzymes.

A compound which is a MEK inhibitor may be determined by using an assay known to one of skill in the art that measures MEK inhibition. For example, MEK inhibition may be determined using the assays titled, “Enzyme Assays” in U.S. Pat. No. 5,525,625, column 6, beginning at line 35. The complete disclosure of U.S. Pat. No. 5,525,625 is hereby incorporated by reference. Specifically, a compound is an MEK inhibitor if a compound shows activity in the assay titled, “Cascade Assay for Inhibitors of the MAP Kinase Pathway,” column 6, line 36 to column 7, line 4 of the U.S. Pat. No. 5,525,625 and/or shows activity in the assay titled, “In Vitro MEK Assay” at column 7, lines 4 to 27 of the above-referenced patent. Alternatively, MEK inhibition can be measured in the assay described in WO 02/06213 A1, the complete disclosure of which is hereby incorporated by reference.

Examples of MEK inhibitors according to the present invention include, but are not limited to the MEK inhibitors disclosed in the following PCT Publications: WO 99/01426, WO 99/01421, WO 00/42002, WO 00/42022, WO 00/41994, WO 00/42029, WO 00/41505, WO 00/42003, WO 01/68619, and WO 02/06213.

A pharmaceutically or therapeutically effective amount or dosage of CI-1040, Compound A or capecitabine may be understood to comprise an amount sufficient to prevent or inhibit the growth of tumor cells or the progression of cancer metastasis in the combinations of the present invention. Therapeutic or pharmacological effectiveness of the doses and administration regimens may also be characterized as the ability to induce, enhance, maintain or prolong remission in patients experiencing specific tumors.

The compounds to be utilized in the methods or combinations of the present invention may be administered in dosages or doses commonly employed clinically. Those skilled in the art will be able to determine, according to known methods, the appropriate therapeutically effective amount or dosage of each compound, as used in the combination of the present invention, to administer to a patient, taking into account factors such as age, weight, general health, the compound administered, the route of administration, the nature and advancement of the cancer requiring treatment, and the presence of other medications. Such doses may be calculated in the normal fashion, for example on body surface area. Alternatively, an effective amount or a therapeutically effective amount may be calculated in mg/kg of body weight. Commercially available capsules, tablets, or other formulations (such as liquids and film-coated tablets) can be administered according to the disclosed methods.

Capecitabine for monotherapy generally is administered orally at a dose of about 2500 mg/m² daily for 2 weeks, followed by a 1-week rest period. The product is supplied commercially in 150 mg and 500 mg tablets. The tablets are administered at the rate of about 1 to about 4 times a day during the treatment period. The daily doses of capecitabine may, for example, range from about 1000 mg/m² to about 3500 mg/m² per day in the combinations of this invention.

CI-1040 for monotherapy generally may be administered until progression of the disease state is observed, for example, CI-1040 may be administered daily from about 2-4 weeks to the duration of the life of the patient. CI-1040 may be administered at doses from about 100 mg to about 1600 mg once a day (“qd”), or from about 400 to about 800 mg two or three times a day (“bid” or “tid”, respectively) with or without food. For example, CI-1040 may be administered at 800 mg twice a day with food. CI-1040 typically is administered orally, for example, as capsules having active ingredient in the amounts of 5, 25, and 200 mg per capsule. Multiple treatment periods can be practiced, as dictated by the attending medical practitioner and the particular patient and condition being treated.

Compound A for monotherapy generally may be administered until progression of the disease state is observed, for example, Compound A may be administered daily from about 2-4 weeks to the duration of the life of the patient. Compound A may be administered at a daily dose range between about 0.1 and about 1000 mg/kg per day, preferably between about 1 and about 300 mg/kg body weight, and daily dosages will be between about 1 and about 500 mg for an adult subject of normal weight, preferably between about 1 mg and 50 mg. For example, Compound A may be administered at a daily dose range may be between about 1 mg and about 20 mg, in a single dosage or in divided doses. According to the disclosed methods, Compound A may be administered orally, for example, as capsules, such as hard gelatin capsules, or other formulations, such as liquids and film-coated tablets having active ingredient in the amounts of, for example, 0.25 mg, 0.5 mg, 1 mg, 5 mg, 10 mg, 25 mg, 50 mg, 100 mg, 200 mg, 300 mg, or 400 mg can be administered. Multiple treatment periods can be practiced, as dictated by the attending medical practitioner and the particular patient and condition being treated.

In some instances, dosage levels below the lower limit of the aforesaid range may be more than adequate, while in other cases still larger doses may be employed, as determined by those skilled in the art.

More particularly, according to the method of the present invention, the effective dosage level of a MEK inhibitor may range from about 5% to about 100% of the effective dosage level when used without capecitabine. In addition, the effective dosage level of capecitabine may range from about 5% to about 100% of the effective dosage level when used without a MEK inhibitor.

In accord with procedures generally known and practiced in the art, when used in combination, the dosage level of capecitabine and the MEK inhibitor may be adjusted to achieve the optimum effective dosage level.

The practice of the methods of this invention may be accomplished through various administration regimens. One method of treating or inhibiting cancer cells or tumors of this invention comprises the contemporaneous or simultaneous administration of pharmaceutically or therapeutically effective amounts of a MEK inhibitor, such as CI-1040 and Compound A, and capecitabine to a patient in need of such treatment. A joint administration of both compounds may be conducted over a period of time deemed appropriate by a medical professional for the recipient in question. One regimen may include administration of both compounds over a period of from 2 to 4 weeks. Repetition of the joint administration may be conducted for a series of dosage periods, as necessary to achieve the desired reduction or diminution of cancer cells. Optionally, the series of joint administration may be separated by non-treatment periods of from, for example, 2 to 6 weeks to allow conventional patient rest and recovery.

Methods of this invention also include administration to a patient in need thereof a pharmaceutically or therapeutically effective amount of CI-1040 or Compound A for or over a specific period or regimen, followed by administration to the patient of a subsequent regimen of a pharmaceutically or therapeutically effective amount of capecitabine. An example of such a regimen would include administration to a patient of a therapeutically or pharmaceutically effective amount of CI-1040 for from 14 to 28 days, followed by administration of a pharmaceutically or therapeutically effective amount of capecitabine for a subsequent and connecting period of from 7 to 14 days. Administration of capecitabine may be separated by non-treatment periods of from, for example, 2 days to a week to allow conventional patient rest and recovery.

Another method of practicing this invention comprises sequential administrations of a regimen of capecitabine administration, followed by a regimen of CI-1040 or Compound A administration. Examples of such a regimen would include an initial administration of a pharmaceutically or therapeutically effective amount of capecitabine for 7 to 14 days with non-treatment periods of from 2 days to a week to allow conventional patient rest and recovery, followed by administration of a therapeutically or pharmaceutically effective amount of CI-1040 for from 14 to 28 days. Repetitive sequences of this type of capecitabine regimen followed by CI-1040 regimen may be continued, as needed, with optional interim periods of non-treatment as determined by a medical professional.

The compounds of the methods or combinations of the present invention may be formulated prior to administration. These compounds may be formulated either separately or in combination with pharmaceutically acceptable carriers as known in the art and administered in a wide variety of dosage forms as known in the art. In making the pharmaceutical compositions of the present invention, the active ingredient will usually be mixed with a carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

MEK inhibitors, such as CI-1040 and Compound A, can be formulated for administration by the oral or parenteral routes. They can also be administered topically, such as transdermally, as skin patches or lotions, or as suppositories. Simultaneous administration of a MEK inhibitor and capecitabine may be by the same (both actives by either local or systemic injection) or different routes. While CI-1040, for example, can be formulated with capecitabine, for instance in solution for intravenous injection or infusion, the active agents will more typically be formulated individually in their normal preparations, and will be administered individually. CI-1040, for example, and capecitabine can be formulated individually and packaged together, in a kit for example, for convenience in usage. Alternatively, the agents can be formulated together in a single formulation, in which case the capecitabine will be present at concentrations ranging from about 1 to about 1000 parts by weight relative to the MEK inhibitor, and the MEK inhibitor will be present at concentrations of about 1000 to about 1 part by weight relative to the capecitabine. Generally, the agents will be administered at about equal doses, or as otherwise approved by health regulatory agencies.

Dosage unit forms can be adapted for various methods of administration, including controlled release formulations, such as subcutaneous implants. Administration methods include oral, rectal, parenteral (intravenous, intramuscular, and subcutaneous), intracisternal, intravaginal, intraperitoneal, intravesical, local (drops, powders, ointments, gels, or cream), and by inhalation (a buccal or nasal spray).

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size.

Additionally, it is also possible to administer the active agents used in accordance with the present invention topically, and this may be done by way of creams, jellies, gels, pastes, patches, ointments and the like, in accordance with standard pharmaceutical practice.

Carriers for solid dosage forms include (a) fillers or extenders, (b) binders, (c) humectants, (d) disintegrating agents, (e) solution retarders, (f) absorption acccelerators, (g) adsorbants, (h) lubricants, (i) buffering agents, and (j) propellants. Pharmaceutical compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

Specific examples of oral formulations of Compound A in hard gelatin capsules may include dosages of the active pharmaceutical agent, for example, from 0.1 mg to 50 mg per capsule. The compositions may include the active drug substance, such as N-[(R)-2,3-Dihydroxy-propoxy]-3,4-difluoro-2-(2-fluoro-4-iodo-phenylamino)-benzamide form IV, a diluent, such as microcrystalline cellulose, and a disintegrant, such as croscarmellose sodium. The composition may also contain a lubricant, such as stearic acid or magnesium stearate.

Examples of these oral formulations in hard gelatin capsules include those in which the active drug substance comprises from about 0.1-20% of the formulation, by weight, a diluent comprises from about 75-95%, a disintegrant comprises from about 3-7% and, optionally, a lubricant comprises from about 0.1-2%.

A 0.25 mg capsule may contain from about 0.15 to about 0.25 % active drug substance, by weight, from about 93-95% microcrystalline cellulose, from about 4-6% croscarmellose sodium and, optionally, from about 0.5-1.5% magnesium stearate.

A 1 mg capsule may contain from about 0.7 to about 0.85 % active drug substance, by weight, from about 92.5-95% microcrystalline cellulose, from about 4-6% croscarmellose sodium and, optionally, from about 0.5-1.5% magnesium stearate.

A 5 mg capsule may contain from about 4% to about 6 % active drug substance, by weight, from about 87-93% microcrystalline cellulose, from about 4-6% croscarmellose sodium and, optionally, from about 0.5-1.5% magnesium stearate.

A 25 mg capsule may contain from about 14% to about 17% active drug substance, by weight, from about 76-83% microcrystalline cellulose, from about 4-6% croscarmellose sodium and, optionally, from about 0.5-1.5% magnesium stearate.

Hard gelatin capsule oral formulation of the type just described may be prepared by methods known in the art. An example includes blending and milling the active drug agent with the desired amount of disintegrant, such as croscarmellose sodium, and half the desired amount of diluent, such as microcrystalline cellulose. The second half of the diluent may then be milled and blended with the first mixture of active agent, diluent and disintegrant and the resulting composition blended. An optional lubricant, such as magnesium stearate, may then be added with additional blending. The total composition may then be measured and placed in hard gelatin capsules. Alternatively, the dry composition may be pressed into slugs using a tablet press, followed by additional milling of the resulting slugs. This final mixture may then be divided into the appropriate dosages and sealed in hard gelatin capsules.

N-[(R)-2,3-Dihydroxy-propoxy]-3,4-difluoro-2-(2-fluoro-4-iodo-phenylamino)-benzamide form IV, can be prepared by a process comprising the steps of:

a) entering an amount of N-[(R)-2,3-Dihydroxy-propoxy]-3,4-difluoro-2-(2-fluoro-4-iodo-phenylamino)-benzamide into a volume of a C₁-C₄ lower alkanol and water, the amount of ethanol to water being at a ratio of from about 1:7 to about 1:13, at a temperature of from above about 30° C. to about 40° C.;

b) stirring the components of step a) to create a mixture of N-[(R)-2,3-Dihydroxy-propoxy]-3,4-difluoro-2-(2-fluoro-4-iodo-phenylamino)-benzamide in alkanol and water;

c) cooling the mixture of N-[(R)-2,3-Dihydroxy-propoxy]-3,4-difluoro-2-(2-fluoro-4-iodo-phenylamino)-benzamide in alkanol and water to a temperature from about 20° C. to less than about 30° C.;

d) separating the N-[(R)-2,3-Dihydroxy-propoxy]-3,4-difluoro-2-(2-fluoro-4-iodo-phenylamino)-benzamide from the alkanol and water.

Within the process parameters discussed above are the steps of preparing polymorphic form IV by:

a) entering an amount of N-[(R)-2,3-Dihydroxy-propoxy]-3,4-difluoro-2-(2-fluoro-4-iodo-phenylamino)-benzamide into a volume of a C₁-C₄ lower alkanol and water, the amount of ethanol to water being at a ratio of from about 1:9 to about 1:11, at a temperature of from about 32° C. to about 38° C.;

b) stirring the components of step a) to create a mixture of N-[(R)-2,3-Dihydroxy-propoxy]-3,4-difluoro-2-(2-fluoro-4-iodo-phenylamino)-benzamide in alkanol and water;

c) cooling the mixture of N-[(R)-2,3-Dihydroxy-propoxy]-3,4-difluoro-2-(2-fluoro-4-iodo-phenylamino)-benzamide in alkanol and water to a temperature from about 22° C. to about 28° C.;

d) separating the N-[(R)-2,3-Dihydroxy-propoxyl-3,4-difluoro-2-(2-fluoro-4-iodo-phenylamino)-benzamide from the alkanol and water.

C₁-C₄ lower alkanols which may be used in this process include methanol, ethanol, propanol, isopropanol, etc., with ethanol being a preferred alkanol. Within the processed described herein is a process in which from about 0.1 to about 5 kg of N-[(R)-2,3-Dihydroxy-propoxy]-3,4-difluoro-2-(2-fluoro-4-iodo-phenylamino)-benzamide are mixed in an alkanol and water mixture having a volume of from about 7.5 to about 15 liters.

### Preparation 1

### N-[(R)-2,3-Dihydroxy-pronoxy]-3,4-difluoro-2-(2-fluoro-4-iodo-]henylamino)-benzamide (Form IV)

To a flask containing 3,4-difluoro-2-(2-fluoro-4-iodo-phenylamino)-benzoic acid (2.6 Kg, 6.6 mol) and N, N′-carbonyldiimidazole (1.1 Kg, 6.8 mol) under nitrogen atmosphere, was added 12 L of dry acetonitrile. After stirring at 22°±5° C. for about 90 minutes, a solution of (R)-O-(2,2-dimethyl-[1,3]dioxolan-4-ylmethyl)-hydroxylamine in toluene was added (8.5 L total volume, about 8 moles of amine). The solution was stirred for at least 6 hours at 22°±5 C. Aqueous hydrochloric acid (9 L, 1.5 molar) was added, and after stirring for about 5 minutes, the layers were separated. Aqueous hydrochloric acid (9 L, 1.5 molar) was added to the remaining top layer, and after stirring for about 20 hours, the layers were separated. The remaining top layer was concentrated by vacuum distillation, and then diluted with 15 L toluene and 2 L ethanol. The mixture was warmed to 35-45° C. and diluted with 20 L warm water, then cooled to 0-5° C. The product was collected by filtration and washed with 2 L toluene. The product was recrystallized by dissolving in 12 L toluene and 2 L ethanol (50°±5 C), adding 10 L water and cooling to 0-5° C. After collecting the product by filtration and washing with toluene, the product was dried in a vacuum oven resulting in 2.6 Kg of N-[(R)-2,3-Dihydroxypropoxy]-3,4-difluoro-2-(2-fluoro-4-iodo-phenylamino)-benzamide. 2.4 Kg of the above compound as a mixture of different crystalline forms was stirred in a mixture of 10 L water and 1 L ethanol at 35±5° C. for 20-30 hours, then cooled to 25+5 C. The product was collected by filtration and washed with 1 L of water, then dried in a vacuum oven at 65° C. This resulted in 2.3 Kg of material which was greater than 90% form IV. Note: DSC analysis shows an onset of melting at 110° C. with only a small amount of the peak with an onset of melting at 117° C.

The following detailed examples further establish the methods of the present invention as described generally above. These examples are illustrative only and are not intended to limit the invention in any way.

### EXAMPLE 1

Tumor Model. The C26/clone 10 mouse colon carcinoma (also referred to as “C26/clone 10 tumor”) was used to evaluate the antitumor activity that was produced when CI-1040 was given in combination with capecitabine. The methods described by Corbett et al. were used for tumor transplantation and the measurement of tumor growth (described below) [Corbett T. et al, “Tumor models and the discovery and secondary evaluation of solid tumor active agents,” Int. J. Pharmacognosy, 1995; 33(supplement): 102-122.; Corbett T H, et al., “The use of rodent tumors in experimental cancer therapy: Conclusions and recommendations,” In: R F Kallman (ed), Rodent models in experimental chemotherapy, (Pergamon Press, 1987),233-247.; Corbett T, Valeriot F, et al. “Use of rodent solid tumors for drug discovery”. In: B A Teicher, (ed), Cancer Drug Discovery, (Human Press Inc., 1997) 75-99. Corbett, T H, et al., “Tumor induction relationships in development of transplantable cancers of the colon in mice of chemotherapy assays, with a note on carcinogen structure,” Cancer Res. 1975; 35(9): 2434-2439; and Corbett, T H, et al., “Evaluation of single agents and combinations of chemotherapeutic agents in mouse colon carcinomas”, Cancer, 1977; 40(5): 2660-2690].

Female Balb/C mice obtained from Charles Rivers Laboratories (Wilmington, Mass.) were used to maintain the tumor and for antitumor testing. These mice are the syngeneic host for the C26/clone 10 tumor. Mice were supplied food and water ad libitum. The average doubling times for the C26/clone 10 tumor in these studies ranged from 3.6 to 4.5 days. Test animals were implanted subcutaneously on day 0 with 30 to 60 mg tumor fragments using a 12 gauge trocar needle. Tumors were measured with a caliper 3 times a week. Tumor weight was calculated from caliper measurements by the following equation:

Tumor weight (mg)=(a×b²)/2,

where “a” is tumor length in millimeters (“mm”) and “b” is tumor width in mm.

On day 7, when the median tumor weights were between 220 and 260 mg, the test animals were randomized into control and treatment groups and chemotherapy was started. These tumor sizes represent an advanced stage of the C26/clone 10 tumor.

Antitumor agents. CI-1040 was suspended in 0.5% hydroxy propyl methyl cellulose and 0.2% Tween-80 in water and administered orally at various dosages in 0.5 mL of the drug suspension. Capecitabine was suspended in 0.5% methyl-cellulose in water and administered orally at various dosage levels in 0.5 mL of the drug suspension.

Dosages and Treatment Schedules. The dosage levels and treatment schedules of the antitumor agents, CI-1040 and capecitabine, were those commonly used in preclinical studies to treat experimental solid tumors. These doses and schedules can be allometrically scaled for humans. CI-1040 was administered orally, three times a day (“tid”), for 14 consecutive days. The doses of CI-1040 were 37.5, 75, 150 and 300 mg/kg/treatment (112.5, 225, 450 and 900 mg/kg/day). Capecitabine was administered orally, once a day (“qd”), for 2 five-day courses with 2 days of rest between the courses. The dosages of capecitabine were 500 and 750 mg/kg/day, with the highest dose being the maximum tolerated dose. Neither drug, given alone at these doses, caused significant weight losses or toxic deaths. For all schedules, treatments were started 7 days after tumor implant when the tumor was an advanced stage.

Measurement of Antitumor Activity. The endpoints used to evaluate antitumor activity were the following: complete and partial tumor responses, tumor growth delay, and the number of tumor-free mice at the end of the study. A complete response was classified as a 100% decrease in tumor mass, and a partial response was classified as at least a 50% decrease in tumor mass. In addition to tumor mass reduction, tumor growth delay (as measured by the methods described by Corbett et al., listed above) was used to quantitate antitumor activity for tumors that did not completely respond, or re-grew after a complete response. Tumor growth delay was expressed as a T−C value, where “T” and “C” are the median time in days required for the treatment group and control group (respectively) tumors to reach a pre-determined size of 750 mg (the “evaluation size”). From the tumor growth delay value the net log₁₀ tumor cell kill was calculated as follows:

Net log₁₀ tumor cell kill=[(T−C)−Rx]/3.32×Td

where “Td” is the number of days for the tumor mass to double and “Rx” is the total days of treatment.

Td was estimated from the best fit straight line of a log-linear plot of the control-group tumors in exponential growth. The conversion of the T−C values to log₁₀ cell kill is possible because the Td for tumors regrowing after treatment is approximately the same as that for untreated control mice. The net log₁₀ kill value normalizes the efficacy data for treatment regimens of varied duration. Positive values indicate that an actual reduction of tumor burden occurred. Negative values indicate the tumor actually grew (although possibly more slowly) during treatment. Tumor-free survivors were excluded from these calculations.

Results. The antitumor activities that were produced when CI-1040 and capecitabine were administered simultaneously are shown in Table 1. In this study, starting on day 7 and ending on day 20, CI-1040 was administered orally, three times a day. The dose of CI-1040 ranged from 37.5 to 300 mg/kg/treatment (112.5 to 900 mg/kg/day). Capecitabine was given orally, once a day, on days 7 through 11, and days 14 through 18. The doses of capecitabine were 500 and 750 mg/kg/day. As shown in Table 1, the vehicle control mice lost 10.5% of their initial body weight during treatment. The C26/clone 10 carcinoma is a highly cachexic tumor and this amount of weight loss was expected. Tumors in vehicle-treated mice grew at a normal rate, and did not differ markedly from the growth of the tumors in the untreated control mice. All animals, that received CI-1040 alone, survived a full course of treatment, and there were no delayed deaths. Over the dosage range of CI-1040 administered, the mice lost approximately 5% of their initial body weight, which is about one half that seen in the vehicle control mice. A lower amount of weight loss in mice bearing the C26/clone 10 colon carcinoma is consistently seen with CI-1040 therapy. CI-1040 alone produced a dose-dependent tumor growth delay that ranged from 3.8 days for the low dose to 16.7 days for the high dose. At 300 mg/kg/treatment, CI-1040 produced no complete and 20% partial tumor responses. Ten percent complete and no partial tumor responses were seen at a dose of 150 mg/kg/treatment. No complete or partial tumor responses were seen with the lower CI-1040 doses. None of the mice were tumor free when the study ended.

All animals that received capecitabine alone survived a full course of treatment, and there were no delayed deaths. Similar to CI-1040, mice treated with capecitabine alone at both doses lost approximately 5% of their initial body weight. Both dosages of capecitabine produced the same tumor growth delay of approximately 18 days. At 500 mg/kg, capecitabine produced 40% complete and 10% partial tumor responses. Of the mice whose tumors completely responded, 30% were still tumor-free when the study ended on day 93. The highest dose of capecitabine produced 70% complete and 10% partial tumor responses. All of the mice that had a complete tumor response were tumor free when the study ended.

As shown in Table 1, CI-1040 could not be given at 150 or 300 mg/kg/treatment with capecitabine at 750mg/kg because of either an unacceptable weight loss, or an unacceptable number of deaths. CI-1040 at its highest dose could also not be given with capecitabine at 500 mg/kg because of an unacceptable number of deaths. CI-1040 at 75 mg/kg/treatment in combination with capecitabine at 750 mg/kg produced 100% complete tumor responses. Sixty percent of these mice were tumor-free when the study ended.

TABLE 1
Antitumor Effect of CI-1040 in Combination with Capecitabine against C26/clone 10 Mouse Carcinoma

| CI-1040 | | Capecitabine | | Non-specific | % Weight | Antitumor Effect | | | | |
|---|---|---|---|---|---|---|---|---|---|---|
| Dose^{a} | Schedule | Dose^{a} | Schedule | Deaths | Change^{b} | CR^{c} | PR^{d} | T-C^{e} (+) | Net Log₁₀ Kill^{f} | Tumor Free^{g} |
| Vehicle | tid, Days 7-20 | Vehicle | qd, Days 7-11, 14-18 | 0/10 | −10.5 | 0/10 | 0/10 | 0 | 0 | 0/10 |
| 37.5 | tid, Days 7-20 | None | None | 0/10 | −5.1 | 0/10 | 0/10 | 3.8 | −0.62 | 0/10 |
| 75.0 | tid, Days 7-20 | None | None | 0/10 | −5.1 | 0/10 | 0/10 | 9.8 | −0.21 | 0/10 |
| 150.0 | tid, Days 7-20 | None | None | 0/10 | −5.1 | 1/10 | 0/10 | 13.5 | 0.03 | 1/10 |
| 300.0 | tid, Days 7-20 | None | None | 0/10 | −5.1 | 0/10 | 2/10 | 16.7 | 0.25 | 0/10 |
| None | None | 500 | qd, Days 7-11, 14-18 | 0/10 | −5.1 | 4/10 | 1/10 | 18.4 | 0.50 | 3/10 |
| None | None | 750 | qd, Days 7-11, 14-18 | 0/10 | −5.1 | 7/10 | 1/10 | 18.2 | 0.48 | 7/10 |
| 37.5 | tid, Days 7-20 | 500 | qd, Days 7-11, 14-18 | 0/10 | −10.0 | 4/10 | 2/10 | 18.4 (22.2) | 0.36 | 2/10 |
| 75.0 | tid, Days 7-20 | 500 | qd, Days 7-11, 14-18 | 0/10 | −10.5 | 4/10 | 1/10 | 18.3 (28.2) | 0.35 | 1/10 |
| 150.0 | tid, Days 7-20 | 500 | qd, Days 7-11, 14-18 | 0/10 | −10.0 | 6/10 | 0/10 | 18.3 (31.9) | 0.35 | 2/10 |
| 300.0 | tid, Days 7-20 | 500 | qd, Days 7-11, 14—18 | 2/10^{h} | −10.0 | 3/10 | 3/10 | 21.2 (35.1) | Toxic | 2/10 |
| 37.5 | tid, Days 7-20 | 750 | qd, Days 7-11, 14-18 | 0/10 | −10.0 | 5/10 | 3/10 | 20.0 (22.0) | 0.47 | 3/10 |
| 75.0 | tid, Days 7-20 | 750 | qd, Days 7-11, 14-18 | 0/10 | −10.5 | 10/10 | 0/10 | 20.1 (28.0) | 0.48 | 6/10 |
| 150.0 | tid, Days 7-20 | 750 | qd, Days 7-11, 14-18 | 0/10 | −15.8 | 10/10 | 0/10 | 24.9 (31.7) | Toxic | 6/10 |
| 300.0 | tid, Days 7-20 | 750 | qd, Days 7-11, 14-18 | 2/10^{h} | −15.8 | 8/10 | 0/10 | 31.5 (34.9) | Toxic | 2/10 |

^{a}Dose is in mg/kg/injection. The vehicle for CI-1040 was composed of 0.5% hydroxy propyl methyl cellulose and 0.2% Tween-80 in water. The vehicle for capcitabine was 0.5% methyl cellulose. Both drugs were dosed orally. Treatment was started when tumors were approximately 250 mg in mass.
^{b}A weight loss is the percent weight loss seen during treatment; the percent weight gain is the weight increase seen at the end of treatment.
^{c}Complete response is defined as a 100% reduction of initial tumor mass.
^{d}Partial response is defined as at least a 50% reduction of initial tumor mass.
^{e}T-C is the difference in days for the treated and control tumors to reach 750 mg. The values in parenthesis represent the T-C values for an additive antitumor effect. All tumor free survivors are excluded from T-C calculations.
^{f}Net log₁₀ tumor cell kill was calculated from the T-C value.
^{g}Tumor free represents the mice that had an undetectable tumor when the study ended on day 93.
^{h}This combination is considered toxic because of an unacceptable number of deaths. The antitumor values for the surviving animals are shown only for comparison
ⁱThis combination is considered toxic because of an unacceptable weight loss. The antitumor values for the surviving animals are shown only for comparison.
^{j}This combination is considered toxic because of an unacceptable weight loss. The antitumor values for the surviving animals are shown only for comparison.

### EXAMPLE 2

Table 2 below shows the antitumor effect that was produced when CI-1040 was administered before capecitabine, according to the procedure of Example 1. CI-1040 was given orally, three times a day at doses of 37.5, 75, 150 and 300 mg/kg/treatment. Consistent with the results of Example 1, the vehicle-control mice bearing the C26/clone 10 mouse colon carcinoma lost 10% of their initial body weight. There was one death in the group of mice that were treated with CI-1040 alone at 300 mg/kg/treatment. (This mouse was found dead on day 19 and had lost 22% of its initial body weight.) This death was not considered to be drug related, but its cause was not known. Also consistent with the results of Example 1, the tumors in vehicle-treated mice grew at a normal rate, and did not differ markedly from the growth of the tumors in the untreated control mice. The remaining mice in this group gained 5.3% in body weight by day 19. No deaths were seen in the other CI-1040 treatment groups, and consistent with Example 1, CI-1040 had an anti-cachexia effect. CI-1040 alone produced a dose-dependent increase in tumor growth delay that ranged from 0.8 to 9.9 days. At 300 mg/kg/treatment, CI-1040 produced no complete and 60% partial tumor responses. No complete or partial tumor responses were seen with the other doses of CI-1040.

One death occurred in each of the two groups treated with capecitabine alone. Like the death seen in the high dose CI-1040 group, these deaths were also unusual because typically, capecitabine at these dosages do not produce deaths. The deaths in the two groups treated with capecitabine alone occurred several days after the last treatment, and their causes are not known. Capecitabine produced a dose-dependent increase in the tumor growth delay that ranged from 10.4 to 19.1 days. At 500 mg/kg/day, capecitabine produced no complete and 10% partial tumor responses. The highest dose of capecitabine produced 50% complete and 20% partial tumor responses. Thirty percent of the mice that had complete tumor responses were still tumor free when the experiment ended on day 41.

When CI-1040 was administered before capecitabine, all dosage combinations of these two drugs were well tolerated. The highest weight losses were no greater that those seen in the vehicle control groups, and in most cases they were less severe than those in this control group. No deaths were seen in any combination group treated with CI-1040 and capecitabine. The lack of deaths in the combination groups supports the notion that the deaths in the single drug groups were not drug related. In the groups were CI-1040 was combined with the low dose of capecitabine, there were no complete or partial tumor responses. The tumor growth delays ranged from 1.2 days for the lowest dose combination to 17.5 days for the high dose combination. In the group treated with highest dose of CI-1040 and the highest dose of capecitabine, there were no complete and 10% partial tumor responses. There were no complete or partial tumor responses in the other combination groups with the 750 mg/kg dose of capecitabine. The tumor growth delays in the combination groups with the high capecitabine dose were similar to those in the combination groups with the low capecitabine dose.

TABLE 2
Antitumor Effect of CI-1040 in Combination with Capecitabine against C26/clone 10 Mouse Carcinoma

| CI-1040 | | Capecitabine | | Non-specific | % Weight | Antitumor Effect | | | | |
|---|---|---|---|---|---|---|---|---|---|---|
| Dose^{a} | Schedule | Dose^{a} | Schedule | Deaths | Change^{b} | CR^{c} | PR^{d} | T-C^{e} (+) | Net Log₁₀ Kill^{f} | Tumor Free^{g} |
| Vehicle | tid, Days 7-20 | Vehicle | qd, Days 7-11, 14-18 | 0/10 | −10 | 0/10 | 0/10 | 0 | 0 | 0/10 |
| 37.5 | tid, Days 7-20 | None | None | 0/10 | 0 | 0/10 | 0/10 | 0.8 | −0.94 | 0/10 |
| 75.0 | tid, Days 7-20 | None | None | 0/10 | +5.3 | 0/10 | 0/10 | 3.7 | −0.72 | 0/10 |
| 150.0 | tid, Days 7-20 | None | None | 0/10 | 0 | 0/10 | 0/10 | 8.6 | −0.34 | 0/10 |
| 300.0 | tid, Days 7-20 | None | None | 1/10 | +5.3 | 0/10 | 6/10 | 9.9 | −0.24 | 0/10 |
| None | None | 500 | qd, Days 7-11, 14-18 | 1/10 | 0 | 0/10 | 1/10 | 10.4 | −0.05 | 0/10 |
| None | None | 750 | qd, Days 7-11, 14-18 | 1/10 | −5.3 | 5/10 | 2/10 | 19.1 | 0.63 | 3/10 |
| 37.5 | tid, Days 7-20 | 500 | qd, Days 21-25, 28-32 | 0/10 | −10.5 | 0/10 | 0/10 | 1.2 (11.2) | −1.84 | 0/10 |
| 75.0 | tid, Days 7-20 | 500 | qd, Days 21-25, 28-32 | 0/10 | −5.3 | 0/10 | 0/10 | 3.4 (14.1) | −1.67 | 0/10 |
| 150.0 | tid, Days 7-20 | 500 | qd, Days 21-25, 28-32 | 0/10 | 0 | 0/10 | 0/10 | 16.0 (19.0) | −0.70 | 0/10 |
| 300.0 | tid, Days 7-20 | 500 | qd, Days 21-25, 28-32 | 0/10 | 0 | 0/10 | 0/10 | 17.5 (20.3) | −0.58 | 0/10 |
| 37.5 | tid, Days 7-20 | 750 | qd, Days 21-25, 28-32 | 0/10 | −5.3 | 0/10 | 0/10 | 1.2 (19.9) | −1.84 | 0/10 |
| 75.0 | tid, Days 7-20 | 750 | qd, Days 21-25, 28-32 | 0/10 | −10.5 | 0/10 | 0/10 | 16.8 (22.8) | −0.63 | 0/10 |
| 150.0 | tid, Days 7-20 | 750 | qd, Days 21-25, 28-32 | 0/10 | 0 | 0/10 | 0/10 | 9.2 (27.7) | −1.22 | 0/10 |
| 300.0 | tid, Days 7-20 | 750 | qd, Days 21-25, 28-32 | 0/10 | 0 | 0/10 | 1/10 | 19.0 (29.0) | −0.46 | 0/10 |

^{a}Dose is in mg/kg/injection. The vehicle for CI-1040 was composed of 0.5% hydroxy propyl methyl cellulose and 0.2% Tween-80 in water. The vehicle for capcitabine was 0.5% methyl cellulose. Both drugs were dosed orally. Treatment was started when tumors were approximately 250 mg in mass.
^{b}A weight loss is the percent weight loss seen during treatment; the percent weight gain is the weight increase seen at the end of treatment.
^{c}Complete response is defined as a 100% reduction of initial tumor mass.
^{d}Partial response is defined as at least a 50% reduction of initial tumor mass.
^{e}T-C is the difference in days for the treated and control tumors to reach 750 mg. The values in parenthesis represent the T-C values for an additive antitumor effect. All tumor free survivors are excluded from T-C calculations.
^{f}Net log₁₀ tumor cell kill was calculated from the T-C value.
^{g}Tumor free represents the mice that had an undetectable tumor when the study ended on day 41.

### EXAMPLE 3

Table 3 below shows the antitumor effect that was produced when treatment with capecitabine was followed by treatment with CI-1040 according to the procedure of Example 1. Consistent with Example 1, there was a 10.5% weight loss produced by the tumor in the vehicle control group. Tumors in vehicle-treated mice grew at a normal rate, and did not differ markedly from the growth of the tumors in the untreated control mice. CI-1040 was well tolerated at all doses. The improvements in mouse weights were not as great as those seen in Examples 1 and 2. The weight losses ranged from 5.3% to 10.5%. There were no complete tumor responses in any of the groups given CI-1040 alone. However, a 40% partial tumor rate was seen in the group treated with the highest dose of CI-1040, and 1.0% response rates were seen in the groups treated with 75 and 150 mg/kg/treatment of CI-1040. No complete or partial tumor responses were seen in the group treated with the lowest dose of CI-1040. CI-1040 produced a dose-dependent increase in the tumor growth delays that ranged from 1.9 days to 12.5 days.

In the two groups treated with capecitabine alone, there were no deaths, and the weight losses were similar to those seen in the groups treated with CI-1040 alone. The 500 mg/kg dose of capecitabine did not produce any complete or partial tumor responses. There were no complete and 40% partial tumor responses in the group treated with 750 mg/kg of capecitabine. The low and high doses of capecitabine produced essentially the same tumor growth delays of 13.4 and 14.6 days, respectively.

Table 3 shows the synergistic effects observed when treatment with capecitabine was followed by treatment with CI-1040. When mice were first treated with 500 mg/kg of capecitabine and then were treated with CI-1040 at doses of 37.5 to 300 mg/kg/treatment, there were no deaths. Also, the weight losses were no greater than those seen in the vehicle control group. The best antitumor activity was seen when treatment with 500 mg/kg of capecitabine was followed by treatments with CI-1040 at either 150 or 300 mg/kg/treatment. In the group that received 150 mg/kg/treatment of CI-1040, there were 40% complete and 10% partial tumor responses. The tumor growth delay produced by this combination was 26.6 days, which is greater than additive. Twenty percent of the mice with a complete tumor response were still tumor free when the experiment ended on day 56. In the group treated with 500 mg/kg of capecitabine followed by treatment with 300 mg/kg/treatment of CI-1040, there were 60% complete and 10% partial tumor responses. The tumor growth delay was 27.9 days, which is also greater than additive. Ten percent of the mice were tumor free when the study ended. In the groups that got lower doses of CI-1040, only a 10% complete response rate was seen when 500 mg/kg of capecitabine was followed by 37.5 mg/kg/treatment of CI-1040. The tumor growth delays, produced by the combinations with 500 mg/kg of capecitabine and either 37.5 or 75 mg/kg/treatment of CI-1040, were better than those produced by either drug alone. Tolerability was not as good in these groups that got 750 mg/kg of capecitabine and either 150 or 300 mg/kg/treatment of CI-1040. With these combinations, there were 10% deaths. However, the weight losses were less than those in the vehicle control group. Tumor shrinkage was seen in all combinations with the high dose of capecitabine. In these combinations, the complete response rates ranged from 20% to 60%, and 10% to 20% of the mice were tumor free when the experiment ended. The partial response tumor response rates ranged from 0% to 40%, and the tumor growth delays ranged from 19.2 to 35.6 days. These tumor growth delays were greater than those produced by either drug alone. The ability of these agents when used together establish the combination to be synergistic as an antitumor agent.

TABLE 3
Antitumor Effect of CI-1040 in Combination with Capecitabine against C26/clone 10 Mouse Carcinoma

| CI-1040 | | Capecitabine | | Non-specific | % Weight | Antitumor Effect | | | | |
|---|---|---|---|---|---|---|---|---|---|---|
| Dose^{a} | Schedule | Dose^{a} | Schedule | Deaths | Change^{b} | CR^{c} | PR^{d} | T-C^{e} (+) | Net Log₁₀ Kill^{f} | Tumor Free^{g} |
| Vehicle | tid, Days 7-20 | Vehicle | qd, Days 7-11, 14-18 | 0/10 | −10.5 | 0/10 | 0/10 | 0 | 0 | 0/10 |
| 37.5 | tid, Days 7-20 | None | None | 0/10 | −5.6 | 0/10 | 0/10 | 1.9 | −0.93 | 0/10 |
| 75.0 | tid, Days 7-20 | None | None | 0/10 | −10.5 | 0/10 | 1/10 | 9.0 | −0.33 | 0/10 |
| 150.0 | tid, Days 7-20 | None | None | 0/10 | −5.6 | 0/10 | 1/10 | 12.3 | −0.05 | 0/10 |
| 300.0 | tid, Days 7-20 | None | None | 0/10 | −5.3 | 0/10 | 4/10 | 12.5 | −0.04 | 0/10 |
| None | None | 500 | qd, Days 7-11, 14-18 | 0/10 | −5.6 | 0/10 | 0/10 | 13.4 | 0.20 | 0/10 |
| None | None | 750 | qd, Days 7-11, 14-18 | 0/10 | −10.5 | 0/10 | 4/10 | 14.6 | 0.30 | 0/10 |
| 37.5 | tid, Days | 500 | qd, Days 7-11, 14-18 | 0/10 | −5.3 | 1/10 | 0/10 | 17.2 (15.3) | −0.82 | 0/10 |
| | 21-34 | | | | | | | | | |
| 75.0 | tid, Days | 500 | qd, Days 7-11, 14-18 | 0/10 | 0 | 0/10 | 0/10 | 18.3 (22.4) | −0.73 | 0/10 |
| | 21-34 | | | | | | | | | |
| 150.0 | tid, Days | 500 | qd, Days 7-11, 14-18 | 0/10 | −10.5 | 4/10 | 1/10 | 26.6 (25.7) | −0.03 | 2/10 |
| | 21-34 | | | | | | | | | |
| 300.0 | tid, Days | 500 | qd, Days 7-11, 14-18 | 0/10 | −5.6 | 6/10 | 1/10 | 27.9 (25.9) | 0.08 | 1/10 |
| | 21-34 | | | | | | | | | |
| 37.5 | tid, Days | 750 | qd, Days 7-11, 14-18 | 0/10 | 0 | 3/10 | 4/10 | 19.2 (16.5) | −0.65 | 2/10 |
| | 21-34 | | | | | | | | | |
| 75.0 | tid, Days | 750 | qd, Days 7-11, 14-18 | 0/10 | +5.6 | 2/10 | 2/10 | 19.9 (23.6) | −0.59 | 1/10 |
| | 21-34 | | | | | | | | | |
| 150.0 | tid, Days | 750 | qd, Days 7-11, 14-18 | 1/10^{h} | −5.6 | 6/10 | 0/10 | 32.7 (26.9) | 0.48 | 2/10 |
| | 21-34 | | | | | | | | | |
| 300.0 | tid, Days | 750 | qd, Days 7-11, 14-18 | 1/10^{h} | −5.6 | 5/10 | 3/10 | 35.3 (27.1) | 0.69 | 1/10 |
| | 21-34 | | | | | | | | | |

^{a}Dose is in mg/kg/injection. The vehicle for CI-1040 was composed of 0.5% hydroxy propyl methyl cellulose and 0.2% Tween-80 in water. The vehicle for capcitabine was 0.5% methyl cellulose. Both drugs were dosed orally. Treatment was started when tumors were approximately 250 mg in mass.
^{b}A weight loss is the percent weight loss seen during treatment; the percent weight gain is the weight increase seen at the end of treatment.
^{c}Complete response is defined as a 100% reduction of initial tumor mass.
^{d}Partial response is defined as at least a 50% reduction of initial tumor mass.
^{e}T-C is the difference in days for the treated and control tumors to reach 750 mg. The values in parenthesis represent the T-C values for an additive antitumor effect. All tumor free survivors are excluded from T-C calculations.
^{f}Net log₁₀ tumor cell kill was calculated from the T-C value.
^{g}Tumor free represents the mice that had an undetectable tumor when the study ended on day 56.
^{h}This combination is considered toxic because of an unacceptable number of deaths. The antitumor values for the surviving animals are shown only for comparison.

### EXAMPLE 4

Tumor Model. COLO-205 human colon carcinoma xenografts were maintained by serial transplantation as subcutaneous implants in female NCr-nu athymic mice. Similar implants were used to evaluate the antitumor action of Compound A and capecitabine. The methods described by Corbett et al. were used for tumor transplantation and the measurement of tumor growth (1-6). Three experiments, described in Examples 4, 5, and 6, were carried out, each employing a different combination treatment regimen. All mice weighed >17 grams at the start of therapy. Mean group weights were well matched within and across the three experiments. Mean group weights at first treatment and associated ranges for Examples 4, 5, and 6 were 21.1(20-22), 22.4(21-24), and 24.2(24-25) grams respectively. Mice were supplied food and water ad libitum. Test animals were implanted subcutaneously on day 0 with 30 to 60 mg tumor fragments using a 12-gauge trocar needle. Tumors were measured with a caliper twice weekly. Tumor weight was calculated from caliper measurements by the following equation:

Tumor weight (mg)=(a×b²)/2,

where “a” and “b” are the respective tumor length and width measurements in mm.

Initial tumor burdens for Examples 4, 5, and 6 were also well matched within and across the three studies. Initial median tumor burdens and associated ranges for the three experiments were 230(221-237), 221(216-270), and 221(216-270) mg respectively. Thus treatment was begun at an advanced tumor stage.

Antitumor agents. Compound A was suspended in 0.5% hydroxypropylmethyl cellulose and 0.2% Tween-80 in water and administered orally (p.o.) in 0.5 ml by gavage. Capecitabine was prepared for injection in 0.5% methylcellulose and administered by gavage.

Measurement of Antitumor Activity. The endpoints used to evaluate antitumor activity were the following: complete and partial tumor responses, tumor growth delay, and the number of tumor-free mice at the end of the study. A complete response was classified as a 100% decrease in tumor mass, and a partial response was classified as at least a 50% decrease in tumor mass. In addition to tumor mass reduction, tumor growth delay (as measured by the methods described by Corbett et al., listed above) was used to quantitate antitumor activity for tumors that did not completely respond, or re-grew after a complete response. Tumor growth delay was expressed as a T−C value, where “T” and “C” are the median time in days required for the treatment group and control group (respectively) tumors to reach a pre-determined size of 750 mg (the “evaluation size”). From the tumor growth delay value the net log₁₀ tumor cell kill was calculated as follows:

Net log₁₀ tumor cell kill=[(T−C)−Rx]/3.32×Td

where “Td” is the number of days for the tumor mass to double and “Rx” is the total days of treatment.

Td was estimated from the best-fit straight line of a log-linear plot of the control-group tumors in exponential growth (200 to 800 mg range). The mean Tds for the control groups Examples 4, 5, and 6 were 8.8, 9, and 11.2 days respectively. Substantial variability in doubling times within an individual experiment was observed. The range of Tds for individual mice was 3.8-15.8, 5.8-13.9, and 5.4-20.1 for Examples 4, 5, and 6 respectively. The conversion of the T−C values to log₁₀ cell kill is valid only if the Td for tumors regrowing after treatment is approximately the same as that for untreated control mice. The net log₁₀ kill value allows quantitative comparison of efficacy across multiple experimental protocols and across models by normalizing the efficacy data for treatment regimens of varied duration and differences in tumor growth rates between experiments or models. Positive values indicate that an actual reduction of tumor burden occurred at the end of therapy relative to the pretreatment burden. Negative values indicate the tumor actually grew (although possibly more slowly than the control tumors) during treatment. Thus negative net kill values do not necessarily imply a complete lack of activity. Tumor-free survivors were excluded from calculations of net kill.

Control tumor growth was within normal bounds for all experiments. Vehicle treated and untreated animals lost between 0 and 9% body weight during treatment, presumably due to progression of the disease and/or dosing related trauma. The results of these studies are summarized in Tables 4-6.

Results. The antitumor activities that were produced when Compound A was administered before capecitabine are shown in Table 4. Compound A was given as a single agent, qd from days 16-29 post tumor implant at doses ranging from 3.13 to 25 mg/kg. The 25 mg/kg level was not tolerated and 12.5 mg/kg was considered the maximum tolerated dose (MTD). Weight loss was generally limited (<5%), occurred early in the treatment regimen, and complete recovery was typically observed during ongoing therapy at doses from 3.13 to 25 mg/kg. Compound A was active against this tumor model, producing >50% complete regressions at all tolerated doses and dose dependent growth delays of up to 42 days at the MTD. Net kill calculations suggest >10% of tumor cells survived treatment at all tolerated dose levels.

Capecitabine was given as a single agent by gavage on days 16-29 post tumor implant, at doses of 500 and 650 mg/kg. Neither dose level was lethal, but a 19% loss of body weight was observed at the 650 mg/kg dose level. The 650 mg/kg dose level was declared the MTD in this experiment. Capecitabine was active against this tumor model in a dose dependent manner, producing tumor regressions and substantial tumor growth delays that suggest an approximate 1-log reduction in tumor burden.

This experiment examined sequential therapy with the MEK inhibitor given prior to a course of capecitabine. Therefore, in the combination regimens, capecitabine was given by gavage on days 30 through 43, while Compound A was given on days 16 through 29. All combination regimens containing 25 mg/kg of Compound A were toxic. All other combination regimens were tolerated (≦LD₁₀ and/or <20% weight loss) and thus were evaluated for efficacy. Because of the high incidence of complete regressions noted in the Compound A single agent arm of the study, complete response (“CR”) and partial response (“PR”) measurements did not provide a useful discriminator between single agent and combination therapy. However the incidence of tumor free survivors 188 days post tumor implant was consistently higher for all combination regimens than found for the corresponding single agent regimens. In addition most combination regimens produced tumor growth delays that were significantly longer than those produced by the best single agent regimens. Four of the six tolerated combination regimens produced net tumor cell kill values that were between 0.1 and 0.3 logs better than optimal single agent therapy. Thus, the sequential combination therapy of administration of Compound A followed by administration of capecitabine appeared be marginally more active than optimal single agent therapy with comparable toxicity. Analysis of the low dose groups suggests that the activity of these two agents is essentially additive on this protocol.

TABLE 4
Antitumor effect against the Colo-205 human colon carcinoma that is produced by treatment
first with a course of Compound A and then by treatment
with a course of capecitabine (Exp. 90432 × 19).

| Compound A | | Capecitabine | | Tolerance | | Antitumor Effect | | | | |
|---|---|---|---|---|---|---|---|---|---|---|
| Dose^{a} | Schedule | Dose^{a} | Schedule | Non-specific Deaths | % Weight Change^{b} | CR^{c} | PR^{d} | T-C^{e} | Net Log₁₀ Kill^{f} | Tumor Free^{g} |
| Vehi- | QD, days 16-29 | None | QD, days 16-29 | 0/10 | 0.0 | 0/10 | 0/10 | None | None | 0/10 |
| cle | | | | | | | | | | |
| 25 | QD, days 16-29 | None | None | 8/10 | Toxic | Toxic | Toxic | Toxic | Toxic | Toxic |
| 12.5 | QD, days 16-29 | None | None | 0/10 | −4.8 (23) | 8/10 | 0/10 | 41.9 | 1.0 | 1/10 |
| 6.25 | QD, days 16-29 | None | None | 0/10 | 0.0 | 9/10 | 1/10 | 30.1 | 0.6 | 1/10 |
| 3.13 | QD, days 16-29 | None | None | 0/10 | 0.0 | 6/10 | 3/10 | 40.6 | 0.9 | 0/10 |
| None | None | 650 | QD, days 16-29 | 0/10 | −19.0 (26) | 3/10 | 4/10 | 56.0 | 1.5 | 2/10 |
| None | None | 500 | QD, days 16-29 | 0/10 | −9.1 (23) | 0/10 | 1/10 | 33.4 | 0.7 | 0/10 |
| 25 | QD, days 16-29 | 650 | QD, days 30-43 | 5/10 | Toxic | Toxic | Toxic | Toxic | Toxic | Toxic |
| 12.5 | QD, days 16-29 | 650 | QD, days 30-43 | 1/10 | −4.8 (20) | 9/10 | 0/10 | 75.4 | 1.7 | 3/10 |
| 6.25 | QD, days 16-29 | 650 | QD, days 30-43 | 0/10 | −13.6 (33) | 9/10 | 1/10 | 69.2 | 1.4 | 3/10 |
| 3.13 | QD, days 16-29 | 650 | QD, days 30-43 | 0/10 | −19.0 (47) | 1/10 | 3/10 | 65.8 | 1.3 | 1/10 |
| 25 | QD, days 16-29 | 500 | QD, days 30-43 | 4/10 | Toxic | Toxic | Toxic | Toxic | Toxic | Toxic |
| 12.5 | QD, days 16-29 | 500 | QD, days 30-43 | 1/10 | −4.5 (20) | 9/10 | 0/10 | 68.6 | 1.4 | 3/10 |
| 6.25 | QD, days 16-29 | 500 | QD, days 30-43 | 0/10 | −4.8 (20) | 10/10 | 0/10 | >78.8 | 1.8 | 3/10 |
| 3.13 | QD, days 16-29 | 500 | QD, days 30-43 | 0/10 | + | 9/10 | 0/10 | 73.3 | 1.6 | 2/10 |

^{a}Doses are in mg/kg. Both drugs were administered orally, once a day, for 14 consecutive days. The vehicle for Compound A was composed of 0.5% hydroxypropylmethylcellulose and 0.2% Tween-80 in water. The vehicle for capecitabine was 0.5% methylcellulose in water. Treatments were started 16 days after tumor implantation, when the median tumor masses were ˜221 mg.
^{b}Maximum treatment related weight loss, expressed as a percent of mean group weight at initial treatment. Value in parentheses indicates the day the maximum weight loss was recorded. A net weight gain is represented by a “+”
^{c}Complete response represents a tumor that decreased in mass to less than 62 mg (limit of detection) during the study.
^{d}Partial response represents a tumor that decreased by is at least a 50% of its initial tumor mass.
^{e}T-C is the difference in days for the treated and control tumors to reach 750 mg. All tumor free survivors are excluded from T-C calculations.
^{f}Net log₁₀ tumor cell kill represents the change in tumor burden during therapy.
^{g}Tumor free represents the mice that had an undetectable tumor when the study ended on day 118.

### EXAMPLE 5

Table 5 below shows the antitumor effect that was produced when treatment with capecitabine was followed by treatment with Compound A according to the procedure of Example 4. Compound A was given as a single agent on days 18-31 post tumor implant at doses ranging from 3.13 to 25 mg/kg. The 25 mg/kg level was not tolerated and 12.5 mg/kg was considered the MTD. Weight loss was generally limited (0-5%), occurred early in the treatment regimen, and complete recovery was typically observed during ongoing therapy at doses from 3.13 to 12.5 mg/kg. Compound A was active against this tumor model, producing complete regressions at all tolerated doses and dose dependent growth delays of up to 50 days. Net kill calculations suggest >10% of tumor cells survived treatment at most tolerated dose levels. The activity in this experiment was comparable to that in Example 4 and, across the dose response, modestly superior to that in Example 6.

Capecitabine was given as a single agent by gavage on days 18-31 post tumor implant, at doses of 500 and 650 mg/kg. Both dose levels were tolerated and 650 mg/kg was declared the MTD in this experiment. Capecitabine was also active in a dose dependent manner, producing tumor regressions and substantial tumor growth delays that suggest an approximate 0.5 log reduction in tumor burden. Activity was generally lower than that observed in experiment Example 4 and comparable to that in Example 6.

This experiment examined sequential therapy with capecitabine given prior to a course of the MEK inhibitor. Therefore, in the combination regimens, capecitabine was given by gavage on days 18-31, while Compound A was given on days 32-45. Many of the combination regimens in this experiment were toxic. At 650 mg/kg of capecitabine, only the low dose of Compound A was tolerated in the combination. Only the 3.13 and 6.25 mg/kg dose levels of Compound A were tolerated in combination with capecitabine at 500 mg/kg. Thus only three combination regimens could be evaluated for efficacy. Two of these produced net cell kill values of 1.5 logs, 0.2 log better than the best single agent activity observed. The incidence of tumor free survivors was not higher for these combination regimens than in the single agent arms of the experiment. Analysis of the low dose groups suggested less than additive activity. Thus the sequential combination of administration of capecitabine followed by the administration of Compound A appeared to offer little benefit compared to optimal use of the most active single agent.

TABLE 5
Antitumor Effect against the Colo-205 human colon carcinoma that is produced by treatment
first with a course of capecitabine and then by treatment
with a course of Compound A.

| Compound A | | Capecitabine | | Tolerance | | Antitumor Effect | | | | |
|---|---|---|---|---|---|---|---|---|---|---|
| Dose^{a} | Schedule | Dose^{a} | Schedule | Non-specific Deaths | % Weight Change^{b} | CR^{c} | PR^{d} | T-C^{e} | Net Log₁₀ Kill^{f} | Tumor Free^{g} |
| None | None | None | None | 0/10 | + | 0/10 | 0/10 | −2.4 | −0.5 | 0/10 |
| Vehi- | qd, days 18-31 | None | qd, days 18-31 | 1/10 | −9.1 (31) | 0/10 | 0/10 | 0.0 | −0.4 | 0/10 |
| cle | | | | | | | | | | |
| 25 | qd, days 18-31 | None | None | 4/10 | Toxic | Toxic | Toxic | Toxic | Toxic | Toxic |
| 12.5 | qd, days 18-31 | None | None | 0/10 | + | 4/10 | 1/10 | 29.1 | 0.5 | 0/10 |
| 6.25 | qd, days 18-31 | None | None | 1/10 | + | −7/10 | 2/10 | 50.7 | 1.3 | 1/10 |
| 3.13 | qd, days 18-31 | None | None | 0/10 | −4.3 (24) | 1/10 | 3/10 | 27.3 | 0.5 | 0/10 |
| None | None | 650 | qd, days 18-31 | 0/10 | −4.3 (24) | 2/10 | 2/10 | 34.3 | 0.7 | 2/10 |
| None | None | 500 | qd, days 18-31 | 1/9 | −9.1 (24) | 1/10 | 1/10 | 26.3 | 0.4 | 1/9 |
| 25 | qd, days 32-45 | 650 | qd, days 18-31 | 7/10 | Toxic | Toxic | Toxic | Toxic | Toxic | Toxic |
| 12.5 | qd, days 32-45 | 650 | qd, days 18-31 | 3/10 | −Toxic | Toxic | Toxic | Toxic | Toxic | Toxic |
| 6.25 | qd, days 32-45 | 650 | qd, days 18-31 | 2/10 | Toxic | Toxic | Toxic | Toxic | Toxic | Toxic |
| 3.13 | qd, days 32-45 | 650 | qd, days 18-31 | 0/10 | −9.1 (24) | 8/10 | 2/10 | 71.4 | 1.5 | 1/10 |
| 25 | qd, days 32-45 | 500 | qd, days 18-31 | 2/10 | Toxic | Toxic | Toxic | Toxic | Toxic | Toxic |
| 12.5 | qd, days 32-45 | 500 | qd, days 18-31 | 2/10 | Toxic | Toxic | Toxic | Toxic | Toxic | Toxic |
| 6.25 | qd, days 32-45 | 500 | qd, days 18-31 | 1/10 | −4.5 (24) | 6/10 | 1/10 | >72.9 | 1.5 | 0/10 |
| 3.13 | qd, days 32-45 | 500 | qd, days 18-31 | 1/10 | −8.7 (24) | 5/10 | 1/10 | 46.4 | 0.6 | 1/10 |

^{a}Doses are in mg/kg. Both drugs were administered orally, once a day, for 14 consecutive days. The vehicle for Compound A was composed of 0.5% hydroxypropylmethylcellulose and 0.2% Tween-80 in water. The vehicle for capecitabine (PD0205015) was 0.5% methylcellulose in water. Treatments were started 18 days after tumor implantation, when the median tumor masses were ˜221 mg.
^{b}Maximum treatment related weight loss, expressed as a percent of mean group weight at initial treatment. Value in parentheses indicates the day the maximum weight loss was recorded. A net weight gain is represented by a “+”
^{c}Complete response represents a tumor that decreased in mass to less than 62 mg (limit of detection) during the study.
^{d}Partial response represents a tumor that decreased by is at least a 50% of its initial tumor mass.
^{e}T-C is the difference in days for the treated and control tumors to reach 750 mg. All tumor free survivors are excluded from T-C calculations.
^{f}Net log₁₀ tumor cell kill represents the change in tumor burden during therapy.
^{g}Tumor free represents the mice that had an undetectable tumor when the study ended on day 112.

### EXAMPLE 6

Table 6 below shows the antitumor activities that were produced when Compound A and capecitabine were administered simultaneously according to the procedure of Example 4.

Compound A was given as a single agent on days 17-30 post tumor implant at doses ranging from 3.13 to 25 mg/kg. The 25 mg/kg level was not tolerated and 12.5 mg/kg was considered the MTD. Weight loss was generally limited (4-8%), occurred early in the treatment regimen, and complete recovery was typically observed during ongoing therapy at doses from 3.13 to 12.5 mg/kg. Compound A was once again active against this tumor model, producing complete regressions at all tolerated doses and dose dependent growth delays of up to 70 days. Net kill calculations suggest <10% of tumor cells survived treatment at the MTD. Tumor burden was essentially held constant at the remaining dose levels. In this experiment activity appeared to fall off rapidly at dose levels- below 12.5 mg/kg. An inspection of the actual tumor growth curves shows that tumor growth at the 12.5 mg/kg level failed to return to control growth rates after treatment, instead leveling off after the tumors reached approximately 500 mg. This complicates the use of net cell kill as an endpoint for the experiment. Overall the activity of Compound A in this experiment was comparable or somewhat lower than that in Examples 4 and 5.

Capecitabine was given as a single agent by gavage on days 17-30 post tumor implant, at doses of 500 and 650 mg/kg. Both dose levels were tolerated and 650 mg/kg was declared the MTD in this experiment. Capecitabine was active in this experiment, producing tumor regressions and substantial tumor growth delays that suggest an approximate 0.5 log reduction in tumor burden. The dose response was inverted in this study with higher activity see at the 500 mg/kg dose level. Overall, capecitabine activity was generally lower than that in experiment Example 4 and comparable to that in Example 5.

This experiment examined simultaneous therapy with capecitabine and PD325901 both given on days 17-30. Many of the combination regimens in this experiment were toxic. Only three combination regimens could be evaluated for efficacy. One of these, 6.25 mg/kg Compound A and 650 mg/kg capecitabine produced 100% complete regressions, a net cell kill value of 1.9 logs, and 40% tumor free survivors on day 129. This is significantly superior activity compared to either of the single agents at their MTDs. The other combination regimens were inferior to optimal single agent therapy.

TABLE 6
Antitumor effect against the Colo-205 human colon carcinoma that
is produced by simultaneous treatment with capecitabine and Compound A

| Compound A | | Capecitabine | | Tolerance | | Antitumor Effect | | | | |
|---|---|---|---|---|---|---|---|---|---|---|
| Dose^{a} | Schedule | Dose^{a} | Schedule | Non-specific Deaths | % Weight Change^{b} | CR^{c} | PR^{d} | T-C^{e} | Net Log₁₀ Kill^{f} | Tumor Free^{g} |
| None | None | None | None | 0/10 | −4.2 (20) | 0/10 | 0/10 | −2.5 | −0.4 | 0/10 |
| Vehi- | qd, days 17-30 | None | qd, days 17-30 | 0/10 | −4.2 (20) | 0/10 | 0/10 | 0 | −0.3 | 1/10 |
| cle | | | | | | | | | | |
| 25 | qd, days 17-30 | None | None | 8/10 | Toxic | Toxic | Toxic | Toxic | Toxic | Toxic |
| 12.5 | qd, days 17-30 | None | None | 0/10 | −8.0 (20) | 9/10 | 1/10 | >69.0 | 1.5 | 1/10 |
| 6.25 | qd, days 17-30 | None | None | 1/10 | −4.2 (20) | 8/10 | 1/10 | 18.1 | 0.1 | 1/10 |
| 3.13 | qd, days 17-30 | None | None | 0/10 | −4.0 (20) | 7/10 | 2/10 | 15.3 | 0.1 | 0/10 |
| None | None | 650 | qd, days 17-30 | 0/10 | −12.5 (27) | 1/10 | 1/10 | 14.0 | 0.0 | 0/10 |
| None | None | 500 | qd, days 17-30 | 0/10 | −8.3 (22) | 3/10 | 0/10 | 43.4 | 0.8 | 0/10 |
| 25 | qd, days 17-30 | 650 | qd, days 17-30 | 8/10 | Toxic | Toxic | Toxic | Toxic | Toxic | Toxic |
| 12.5 | qd, days 17-30 | 650 | qd, days 17-30 | 3/10 | Toxic | Toxic | Toxic | Toxic | Toxic | Toxic |
| 6.25 | qd, days 17-30 | 650 | qd, days 17-30 | 0/10 | −4.2 (20) | 10/10 | 0/10 | >84.0 | 1.9 | 4/10 |
| 3.13 | qd, days 17-30 | 650 | qd, days 17-30 | 0/10 | −8.0 (27) | 7/10 | 3/10 | 32.7 | 0.5 | 1/10 |
| 25 | qd, days 17-30 | 500 | qd, days 17-30 | 10/10 | Toxic | Toxic | Toxic | Toxic | Toxic | Toxic |
| 12.5 | qd, days 17-30 | 500 | qd, days 17-30 | 2/10 | Toxic | Toxic | Toxic | Toxic | Toxic | Toxic |
| 6.25 | qd, days 17-30 | 500 | qd, days 17-30 | 2/10 | Toxic | Toxic | Toxic | Toxic | Toxic | Toxic |
| 3.13 | qd, days 17-30 | 500 | qd, days 17-30 | 1/10 | −8.3 (44) | 2/10 | 7/10 | 16.2 | 0.1 | 0/10 |

^{a}Doses are in mg/kg. Both drugs were administered orally, once a day, for 14 consecutive days. The vehicle for Compound A was composed of 0.5% hydroxypropylmethylcellulose and 0.2% Tween-80 in water. The vehicle for capecitabine (PD0205015) was 0.5% methylcellulose in water. Treatments were started 17 days after tumor implantation, when the median tumor masses were ˜221 mg.
^{b}Maximum treatment related weight loss, expressed as a percent of mean group weight at initial treatment. Value in parentheses indicates the day the maximum weight loss was recorded.
^{c}Complete response represents a tumor that decreased in mass to less than 62 mg (limit of detection) during the study.
^{d}Partial response represents a tumor that decreased by is at least a 50% of its initial tumor mass.
^{e}T-C is the difference in days for the treated and control tumors to reach 750 mg. All tumor free survivors are excluded from T-C calculations.
^{f}Net log₁₀ tumor cell kill represents the change in tumor burden during therapy. A negative value indicates a net increase in tumor mass during therapy, while a positive value indicates a net tumor burden reduction during therapy. Values near zero indicate tumor stasis during therapy.
^{g}Tumor free represents the mice that had an undetectable tumor when the study ended on day 129.

What is claimed is:

1. A method for treating cancer in a patient in need of such treatment, the method comprising administering to the patient a combination of a therapeutically effective amount of a MEK inhibitor and a therapeutically effective amount of capecitabine.

2. The method of claim 1, wherein the MEK inhibitor and capecitabine are administered simultaneously.

3. The method of claim 1, wherein the MEK inhibitor is administered before capecitabine.

4. The method of claim 1, wherein capecitabine is administered before the MEK inhibitor.

5. The method of claim 1 wherein the cancer is brain, breast, lung, non-small cell lung, ovarian, pancreatic, prostate, renal, colon, cervical, acute leukemia, gastric, melanoma or combinations thereof.

6. The method of claim 1, wherein the MEK inhibitor is CI-1040.

7. The method of claim 2, wherein the MEK inhibitor is CI-1040.

8. The method of claim 3, wherein the MEK inhibitor is CI-1040.

9. A method for treating cancer in a patient in need of such treatment, the method comprising the steps of administering to the patient a therapeutically effective amount of capecitabine followed by administering to the patient a therapeutically effective amount of CI-1040.

10. The method of claim 9, wherein the cancer is brain, breast, lung, non-small cell lung, ovarian, pancreatic, prostate, renal, colon, cervical, acute leukemia, gastric, melanoma, or combinations thereof.

11. The method of claim 1, wherein the MEK inhibitor is N-[(R)-2,3-dihydroxy-propoxy]-3,4-difluro-2-(2-fluoro-4-iodo-phenylamino)-benzamide.

12. The method of claim 2, wherein the MEK inhibitor is N-[(R)-2,3-dihydroxy-propoxy]-3,4-difluro-2-(2-fluoro-4-iodo-phenylamino)-benzamide, wherein CI-1040 is administered before capecitabine.

13. The method of claim 4, wherein the MEK inhibitor is N-[(R)-2,3-dihydroxy-propoxy]-3,4-difluro-2-(2-fluoro-4-iodo-phenylamino)-benzamide, wherein capecitabine is administered before CI-1040.

14. A method for treating cancer in a patient in need of such treatment, the method comprising the steps of administering to the patient a therapeutically effective amount of N-[(R)-2,3-dihydroxy-propoxy]-3,4-difluro-2-(2-fluoro-4-iodo-phenylamino)-benzamide followed by administering to the patient a therapeutically effective amount of capecitabine.

15. The method of claim 14, wherein the cancer is brain, breast, lung, non-small cell lung, ovarian, pancreatic, prostate, renal, colon, cervical, acute leukemia, gastric, melanoma or combinations thereof.
